(19) United States
(12) Patent Application Publication
(10) Pub. No.: US 2025/0074954 A1
(43) Pub. Date: Mar. 6, 2025

(54) NEW JUNIPER POLLEN PROTEIN

(71) Applicant: TAIHO PHARMACEUTICAL CO., LTD., Tokyo (JP)

(72) Inventor: Yuki TANAKA, Ibaraki (JP)

(73) Assignee: TAIHO PHARMACEUTICAL CO., LTD., Tokyo (JP)

(21) Appl. No.: 18/699,112

(22) PCT Filed: Aug. 19, 2022
(86) PCT No.: PCT/JP2022/031310

§ 371 Date: Nov. 4, 2024

(30) Foreign Application Priority Data
Oct. 7, 2021 (JP) 2021-165659

(51) Int. Cl. C07K 14/415; A61K 39/00; A61P 37/04; C07K 16/16; C12N 15/63; C12P 21/02; G01N 33/68

(52) U.S. Cl. CPC C07K 14/415; A61P 37/04; C07K 16/16; C12N 15/63; C12P 21/02; G01N 33/6854; A61K 39/00; G01N 2800/24

(57) ABSTRACT

The present disclosure relates to a juniper pollen protein selected from (a)-(c) below. (a) A protein comprising an amino acid sequence represented by SEQ ID NO: 2. (b) A protein comprising an amino acid sequence represented by SEQ ID NO: 2 with one or several amino acids substituted, deleted, or added, and having a juniper pollen allergen activity. (c) A protein having at least 90% amino acid identity with the amino acid sequence represented by SEQ ID NO: 2, and having a juniper pollen allergen activity.

### TECHNICAL FIELD

The present disclosure relates to a novel protein derived from juniper pollen and to use of the protein.

### BACKGROUND ART

Pollinosis is an allergic disease caused by inhalation of airborne pollen and presents with symptoms such as allergic conjunctivitis (e.g., itchy and painful eyes), rhinitis, skin inflammation, and asthma. Plants belonging to the Cupressaceae family are a major cause of pollinosis worldwide, and in Japan, pollinosis caused by Japanese cedar (genus Cryptomeria) and Japanese cypress (genus Chamaecyparis) is well known (Non-patent literature 1).

Meanwhile, in the United States, Juniperus ashei, a member of the genus Juniperus of the Cupressaceae family, is a major cause of pollinosis, and measures such as ban on tree planting have been implemented (Non-patent literature 2). In Japan, it has also been reported that 26.5% of allergic patients are sensitized to Juniperus rigida belonging to the genus Juniperus, in areas where many Juniperus rigida are distributed. Therefore, it is suggested that plants of the genus Juniperus that cause pollinosis are allergens that should be emphasized as much as Japanese cedar and Japanese cypress (Non-patent literature 3).

Antihistamines, steroidal anti-inflammatory drugs, antileukotrienes, degranulation inhibitors, Th2 cytokine inhibitors, etc. are used to treat these allergic diseases, but all remain symptomatic treatments. In recent years, however, allergen immunotherapy such as subcutaneous allergen immunotherapy (SCIT) and sublingual allergen immunotherapy (SLIT) have been developed one after another and have been approved by the pharmaceutical authorities. Allergen immunotherapy is a treatment that controls the immune response to allergens by administering small doses of allergens to the body. At present, it is believed to be the only method that can cure allergic diseases, and treatment options for pollinosis are increasing.

Research and understanding of the causative allergens are essential for the development of allergen immunotherapy. In the United States, highly antigenic allergens called Jun a 1, Jun a 2, and Jun a 3 have been reported in Juniperus ashei pollen (Non-patent literature 4, 5, and 6) and they have already been cloned. However, SCIT and SLIT drugs using Juniperus ashei allergens have not yet been commercialized. To realize a highly effective allergen immunotherapy for pollinosis caused by the genus Juniperus, a novel allergen protein is needed.

In addition, cross-reactivity has been reported for antigens contained in the pollen of the Cupressaceae family. However, it has been reported that SCIT and SLIT against Japanese cedar pollen are often unsuccessful against Japanese cypress pollinosis (Non-patent literature 7 and 8), suggesting that allergen immunotherapy requires use of an allergen specific to the pollen that causes the allergy. Therefore, there is a high medical need for a novel allergen immunotherapy against the genus Juniperus using an allergen specific to the genus Juniperus.

### CITATION LIST

### Non-Patent Literature

Non-patent literature 1: Gabriella Di Felice, et al., Int Arch Allergy Immunol., 126:280-289, 2001
Non-patent literature 2: Midoro-Horiuti T, et al., J Allergy Clin Immunol., 104:608-612, 1999
Non-patent literature 3: Tetsuo Oka, Jpn. J. Allergol., vol. 43, No. 2-2, 301, 1994
Non-patent literature 4: Midoro-Horiuti T, et al., J. Allergy Clin. Immunol., 104:613-617, 1999
Non-patent literature 5: Yokoyama, et al., Biochem. Biophys. Res. Commun. 275.1, 195-202, 2000
Non-patent literature 6: Midoro-Horiuti, et al., J. Immunol., 164, 2188-2192, 2000
Non-patent literature 7: Atsushi Yuda, Japanese Journal of Rhinology, vol. 54, no. 4, 503-508, 2015
Non-patent literature 8: Atsushi Yuda, J. Otolaryngol. Jpn., vol. 120, no. 6, 833-840, 2017

### SUMMARY OF INVENTION

### Technical Problem

The present disclosure relates to providing a novel juniper pollen protein, and agents, etc. using the same for diagnosing, preventing, and treating allergic diseases caused by juniper pollen.

### Solution to Problem

The present disclosure relates to the discovery of a new protein from a juniper pollen crude antigen, which is highly reactive with lymphocytes and serum IgE from a patient with Japanese cypress pollinosis, and which is useful as an agent for diagnosing, preventing, or treating allergic diseases caused by juniper pollen.

Thus, one aspect of the present disclosure includes the following.

[1] A juniper pollen protein selected from (a)-(c) below:
(a) a protein comprising the amino acid sequence represented by SEQ ID NO:2;
(b) a protein comprising the amino acid sequence represented by SEQ ID NO:2 with one or several amino acids substituted, deleted or added, and having a juniper pollen allergenic activity; and
(c) a protein comprising an amino acid sequence having 90% or more identity to the amino acid sequence represented by SEQ ID NO:2, and having a juniper pollen allergenic activity.
   [2] A polynucleotide encoding a juniper pollen protein selected from (a)-(c) below:
(a) a protein comprising the amino acid sequence represented by SEQ ID NO:2;
(b) a protein comprising the amino acid sequence represented by SEQ ID NO:2 with one or several amino acids substituted, deleted or added, and having a juniper pollen allergenic activity; and
(c) a protein comprising an amino acid sequence having 90% or more identity to the amino acid sequence represented by SEQ ID NO:2, and having a juniper pollen allergenic activity.
   [3] A polynucleotide selected from (d)-(f) below:
(d) a polynucleotide comprising the nucleotide sequence represented by SEQ ID NO: 1;
(e) a polynucleotide that hybridizes with a polynucleotide comprising a nucleotide sequence complementary to the nucleotide sequence represented by SEQ ID NO: 1 under stringent conditions and that encodes a protein having a juniper pollen allergenic activity; and
(f) a polynucleotide that comprises a nucleotide sequence having 90% or more identity to the nucleotide sequence represented by SEQ ID NO: 1 and that encodes a protein having a juniper pollen allergenic activity.
   [4] A recombinant vector comprising the polynucleotide according to [3].
   [5] A transformant comprising the recombinant vector according to [4].
   [6] A method for producing a juniper pollen protein, the method comprising culturing the transformant according to [5] and collecting the juniper pollen protein from the resulting culture.
   [7] An agent for preventing or treating an allergic disease caused by juniper pollen, the agent comprising the juniper pollen protein according to [1] as an active ingredient.
   [8] An agent for preventing or treating an allergic disease caused by Japanese cedar pollen and/or Japanese cypress pollen, the agent comprising the juniper pollen protein according to [1] as an active ingredient.
   [9] An agent for diagnosing an allergic disease caused by juniper pollen, the agent comprising the juniper pollen protein according to [1] as an active ingredient.
   [10] A kit for diagnosing an allergic disease caused by juniper pollen, the kit comprising the juniper pollen protein according to [1] as an active ingredient.
   [11] A method for detecting an allergic disease caused by juniper pollen, the method comprising reacting the juniper pollen protein according to [1] with a sample.
   [12] An agent for diagnosing an allergic disease caused by Japanese cedar pollen and/or Japanese cypress pollen, the agent comprising the juniper pollen protein according to [1] as an active ingredient.
   [13] An antibody against the juniper pollen protein according to [1].
   [14] The antibody against the juniper pollen protein according to [13], wherein the antibody is a monoclonal antibody.
   [15] Use of the juniper pollen protein according to [1] for the manufacture of an agent for preventing or treating an allergic disease caused by juniper pollen.
   [16] Use of the juniper pollen protein according to [1] for the manufacture of an agent for diagnosing an allergic disease caused by juniper pollen.
   [17] The juniper pollen protein according to [1] for preventing or treating an allergic disease caused by juniper pollen.
   [18] The juniper pollen protein according to [1] for diagnosing an allergic disease caused by juniper pollen.
   [19] A method for preventing or treating an allergic disease caused by juniper pollen, the method comprising administering the juniper pollen protein according to [1] to a patient.
   [20] A method for diagnosing an allergic disease caused by juniper pollen, the method comprising administering the juniper pollen protein according to [1] to a patient.

In addition, another aspect of the present disclosure include the following.

[21] A kit for detecting a juniper pollen protein, the kit comprising the antibody according to either one of [13] and [14] as an active ingredient.
[22] A method for detecting a juniper pollen protein, the method comprising detecting a juniper pollen protein by reacting the antibody according to either one of [13] and [14] with the juniper pollen protein.

### Advantageous Effects of Invention

A juniper pollen protein of the present disclosure can be used in an agent for diagnosing, preventing, or treating an allergic disease caused by juniper pollen, and the like.

### BRIEF DESCRIPTION OF DRAWINGS

FIG. 1 A figure showing a crude extract and a purified protein of juniper pollen analyzed by SDS-polyacrylamide gel electrophoresis (SDS-PAGE).

FIG. 2 A figure showing results of measuring proliferative response to a purified juniper protein using peripheral blood mononuclear cells from two subjects with tree allergy symptoms (pollinosis symptoms) in the spring season and one healthy subject.

FIG. 3 A figure showing results of flow cytometry. Basophils in the peripheral blood of nine subjects with tree allergy symptoms in the spring season were activated by a purified recombinant protein and CD203c expression was enhanced. In contrast, CD203c expression remained unchanged in basophils in the peripheral blood of two healthy subjects.

### DESCRIPTION OF EMBODIMENTS

### Juniper Pollen Protein

A juniper pollen protein of the present disclosure is a protein selected from (a)-(c) below:
(a) a protein comprising the amino acid sequence represented by SEQ ID NO:2;
(b) a protein comprising the amino acid sequence represented by SEQ ID NO:2 with one or several amino acids substituted, deleted or added, and having a juniper pollen allergenic activity; and
(c) a protein comprising an amino acid sequence having 90% or more identity to the amino acid sequence represented by SEQ ID NO:2, and having a juniper pollen allergenic activity.

The juniper pollen protein of the present disclosure may comprise a protein comprising the amino acid sequence represented by SEQ ID NO:2 with one or several amino acids substituted, deleted, or added, as long as the protein has a juniper pollen allergenic activity ((b) above). Examples of a juniper pollen protein comprising such an amino acid sequence include an isoform and the like of a juniper pollen protein having the amino acid sequence represented by SEQ ID NO:2.

Here, “one or several amino acids” to be deleted, substituted, or added refer, for example, to 1-10, more preferably 1-5 amino acids. The above addition or deletion also include an addition or a deletion of one to several amino acids at either end.

As one specific example, an isoform includes, for example, deletion of four residues at the C-terminus.

Herein, a “juniper pollen allergenic activity” comprises not only the activity of binding to IgE on mast cells and causing an immediate allergic reaction in an atopic human (De Weck, A L. et al., Int. Arch. Allergy Immunol., 146:177-189, 2008), but also simply the activity of binding to IgE in serum. The activity of binding to IgE can be measured using the procedure described in the pamphlet of International Publication WO2012/105541, etc.

In addition, the juniper pollen protein of the present disclosure may comprise a protein comprising a protein having 90% or more identity to the amino acid sequence represented by SEQ ID NO:2 when its sequence is appropriately aligned in a corresponding manner with the sequence represented by SEQ ID NO:2, as long as the protein has a juniper pollen allergenic activity ((c) above).

Here, the identity to the amino acid sequence represented by SEQ ID NO:2 is preferably 95% or more, more preferably 98% or more. For example, amino acid sequence identity can be calculated using BLAST (Basic Local Alignment Search Tool at the National Center for Biological Information) and setting optional parameters to default values.

The juniper pollen protein of the present disclosure may also form a fusion with a sequence useful for purification such as multi-histidine residues, a protein to ensure stability during recombinant production, or the like.

The juniper pollen protein of the present disclosure can be obtained by an artificial process. For example, the juniper pollen protein of the present disclosure can be obtained as a recombinant protein. For example, the juniper pollen protein of the present disclosure can be obtained by artificially extracting, isolating, and purifying it from pollen.

Known plants belonging to the genus Juniperus include, but are not limited to, mountain cedar (Juniperus ashei), Chinese juniper (Juniperus chinensis), common juniper (Juniperus communis), and shore juniper (Juniperus conferta).

### Polynucleotide Encoding Juniper Pollen Protein

A polynucleotide of the present disclosure encodes the above juniper pollen protein and suitably comprise: (d) a polynucleotide comprising the nucleotide sequence represented by SEQ ID NO: 1; (e) a polynucleotide that hybridizes with a polynucleotide comprising a nucleotide sequence complementary to the nucleotide sequence represented by SEQ ID NO: 1 under stringent conditions and that encodes a protein having a juniper pollen allergenic activity; or (f) a polynucleotide that comprises a nucleotide sequence having 90% or more identity to the nucleotide sequence represented by SEQ ID NO: 1 and that encodes a protein having a juniper pollen allergenic activity.

Polynucleotides (e) and (f) comprise mutants of polynucleotide (d). Such mutants include naturally occurring allelic mutants and non-naturally occurring mutants that can be generated using mutagenesis techniques well known in the field.

The polynucleotide of the present disclosure comprises not only double-stranded DNA, but also various single-stranded DNA and RNA, such as sense and antisense strands constituting the double-stranded DNA. The antisense strand can be used as a probe or the like. DNA includes isolated cDNA which may be obtained, for example, by cloning, a chemical synthesis technique, or a combination thereof. DNA also includes genomic DNA. Furthermore, in addition to the nucleotide sequence encoding the polypeptide of the present disclosure, a nucleotide sequence such as an untranslated region (UTR) sequence or a vector sequence (including an expression vector sequence) may be added to the polynucleotide of the present disclosure.

Here, stringent conditions include, for example, those described in Molecular Cloning: A Laboratory Manual (Fourth Edition, J. Sambrook et. al., 2012). Specifically, hybridization conditions may include: maintaining the temperature at 65° C. for 8-16 hours together with a probe in a solution containing 6×SSC (composition of 1×SSC: 0.15 M sodium chloride, 0.015 M sodium citrate, pH 7.0), 0.5% SDS, 5×Denhardt, and 100 mg/mL herring sperm DNA.

Moreover, the identity to the nucleotide sequence represented by SEQ ID NO: 1 is 90% or more, preferably 95% or more, and more preferably 98% or more. For example, nucleotide sequence identity can be calculated using BLAST and setting optional parameters to default values.

Examples of mutants of the above polynucleotide include those having the 86th g mutated to t, the 154th g mutated to c, the 242nd g mutated to c, the 265th a mutated to g, the 278th c mutated to a, the 289th a mutated to g, the 341st c mutated to t, the 424th c mutated to t, the 454th g mutated to a, the 461st a mutated to g, the 466th c mutated to t, the 484th c mutated to a, the 490th a mutated to c, the 625th g mutated to a, the 645th c mutated to t, the 727th t mutated to c, the 763rd a mutated to g, the 799th a mutated to t, the 907th g mutated to a, the 988th c mutated to t, the 1154th c mutated to t, the 1156th g mutated to a, the 1253rd g mutated to a, or the 1439th a mutated to t in the nucleotide sequence represented by SEQ ID NO:1, where they have at least one of these mutations.

### Obtaining Polynucleotide Encoding Juniper Pollen Protein

A polynucleotide encoding the juniper pollen protein of the present disclosure can be cloned from juniper pollen, and examples of the cloning method include those using known means such as a shotgun method and a PCR method.

For example, a probe that specifically hybridizes with a part of the polynucleotide sequence of the present disclosure can be prepared and then this probe can be used to perform screening against genomic DNA library or cDNA library. Such a probe may be of any sequence and length as long as it specifically hybridizes with at least a part of the polynucleotides of the present disclosure or the complementary strand thereof. In another method, the polynucleotide is synthesized in an artificial way. (Kosuri S. et al., Nature Methods 11, 499-507, 2014)

The polynucleotide of the present disclosure can also be obtained by using a suitable principle to obtain a sequence that hybridizes with a polynucleotide containing a part or the entire polynucleotide of the present disclosure. Examples of such a method include a PCR method using polynucleotides containing a part of the above polynucleotide of the present disclosure as primers, and a method using a polynucleotide containing a part of the above polynucleotide of the present disclosure as a probe.

For example, according to a method that employs amplification means such as PCR, primers are prepared from the 5′- and 3′-sequences (or complementary sequences thereof) of the polynucleotide of the present disclosure, respectively, and these primers are used to amplify the DNA region between these primers by an amplification reaction such as PCR using genomic DNA (or cDNA) or the like as a template, thereby obtaining DNA fragments containing the polynucleotide in a large amount.

The polynucleotide provided in the present disclosure can also be prepared by modifying a polynucleotide comprising the nucleotide sequence represented by SEQ ID NO: 1, for example, by a planned or random mutation method or the like.

Here, the mutation introduced for a planned mutation can be planned, for example, by referring to a characteristic sequence on the polynucleotide sequence. Examples of the method for introducing a random mutation include a PCR method and a method using a mutagen treatment. Examples of the method of introducing a mutation in a planned manner include site-directed mutagenesis, which, more specifically, can be performed using, for example, Site-Directed Mutagenesis System Mutan-Super Express Km kit (Takara Bio), etc. Alternatively, recombinant PCR method (PCR protocols, Academic Press, New York, 1990) can also be used.

### Production of Juniper Pollen Protein

The juniper pollen protein of the present disclosure can be obtained by separation/purification from juniper pollen. While the separation/purification method is not particularly limited, a juniper pollen extract can be separated/purified using a conventionally known method such as gel filtration, ion exchange chromatography, affinity chromatography, etc.

Examples of the pollen source include, but are not limited to, mountain cedar (Juniperus ashei), Chinese juniper (Juniperus chinensis), common juniper (Juniperus communis), and shore juniper (Juniperus conferta). Preferably, the pollen source is mountain cedar (Juniperus ashei).

In addition, a recombinant vector, which is prepared by incorporating the polynucleotide of the present disclosure into a suitable vector, may be introduced into a host cell for intracellular or extracellular expression and collection of a juniper pollen protein.

The vector into which the polynucleotide of the present disclosure is inserted is not particularly limited, as long as it can replicate in the above-mentioned host, and can be appropriately determined according to the type of the host used for the introduction, the introduction method, etc.

Examples of the vector include plasmid DNA, phage DNA, and a viral vector. The vector DNA used to construct the expression vector is widely available and easily accessible. Examples include pUC19 (Takara Bio), pTV118N (Takara Bio), pMAMneo (Clontech), pGEX (GE HealthCare), pET160 (Invitrogen), pDEST (Invitrogen), pIEx (Merck Millipore), and pBacPAK (Clontech). Examples of the viral vector include a baculovirus vector, a retrovirus vector, a lentiviral vector based on human immunodeficiency virus (HIV) or the like, an adenovirus vector, an adeno-associated virus vector (AAV vector), and a DNA or RNA virus such as herpesvirus, vaccinia virus, Pox virus, poliovirus, Sindbis virus, Sendai virus, and simian virus-40 (SV-40).

The host is not particularly limited as long as it is a living cell capable of transformation, and examples thereof include a bacterium such as E. coli or Bacillus subtilis, a fungus such as yeast or filamentous fungus, a cultured insect cell such as Sf9 cell, an insect such as a silkworm, an animal cell, a plant, and a plant-derived cell.

Transformation of the host using the recombinant vector can be performed using protoplast method, competent cell method, electroporation, or the like. The resulting transformant can be cultured under appropriate conditions in a medium containing a carbon source, nitrogen source, metal salt, vitamin, etc. that can be assimilated. The protein can be collected and purified from the thus obtained culture medium by a general method, thereby obtaining a juniper pollen protein of the present disclosure (Sambrook et al., Molecular Cloning: A Laboratory Manual, Fourth Edition, Cold Spring Harbor Laboratory Press, 2012). For example, when E. coli is used as the host, the method described in pET System Manual, 10th edition (Novagen), etc. can be employed.

### Agent for Preventing or Treating Allergic Disease Caused by Juniper Pollen

The juniper pollen protein of the present disclosure can serve as an agent for preventing or treating an allergic disease caused by juniper pollen and can be administered to a human (patient) in need thereof to prevent or treat the allergic disease caused by juniper pollen.

Here, the allergic disease caused by juniper pollen may be any allergic disease caused by an antigen specific to juniper pollen, and specific examples thereof include atopic bronchial asthma, allergic rhinitis, allergic conjunctivitis, and atopic dermatitis.

Such an agent for preventing or treating an allergic disease caused by juniper pollen can be used, for example, as an agent for desensitization therapy for an allergic disease caused by juniper pollen.

The juniper pollen protein of the present disclosure is a protein having an amino acid sequence different from Cry j 1, Cry j 2, Cry j 3, Cha o 1, and Cha o 2, which have been reported as major allergens associated with the development of Japanese cedar pollinosis and Japanese cypress pollinosis based on the results from an amino acid homology analysis using GENETYX Ver. 12, and is an allergen different from the previously known allergens, Cry j 1, Cry j 2, Cry j 3, Cha o 1, and Cha o 2. Thus, the juniper pollen protein of the present disclosure can be combined with the above known proteins to provide a more useful desensitization treatment. In addition, since the juniper pollen protein of the present disclosure is different from the known proteins, it is possible to detect a new allergic patients caused by juniper pollen that is not detectable by the known proteins.

As a result of BAT (Basophil Activation Test), which measures the phenomenon induced in basophil cells by IgE binding, the juniper pollen protein of the present disclosure showed positive response in all subjects who exhibit tree allergy symptoms in the spring season (Example 2). This was clearly higher than the IgE binding of known pollen proteins, Jun a 1 (71.4%, Non-patent literature 2) and Jun a 3 (42.9%, Non-patent literature 6). As for Jun a 2, the IgE binding positivity of its homologue, Cha o 2, was reported to be 82.5% (Allergology International, 70 [2021]: 281-290). Therefore, an agent for preventing or treating an allergic disease caused by juniper pollen, which uses the juniper pollen protein of the present disclosure, has the potential to be applied to a wider range of patients and may be a useful means for solving the existing problems.

Furthermore, in order to increase the response rate in allergen immunotherapy, it is useful to identify a protein that may be an allergen for an individual patient and sensitize the patient using this protein. Traditionally, allergens have been identified based on the detection of specific IgE against total extracts containing allergenic and non-allergenic components extracted from an allergenic material, but more recently, molecular or component-resolved diagnostics (CRD) have been used, in which an IgE antibody is detected against individual molecules (allergen component) to which the patient is sensitized, in other words, the allergen is analyzed and diagnosed at the level of the causative protein.

According to CRD, the individual IgE profiles and allergen patterns allow more detailed testing for cross-reactivity, risk molecules, and prognostically significant sensitization (Curr Allergy Asthma Rep (2013) 13:110-117), and optimized allergen immunotherapy can be provided for each individual patient. Therefore, the provision of the novel allergenic protein of the present disclosure is of great significance in that effective allergen immunotherapy can be achieved for a larger number of allergic patients. In this case, the allergenic protein does not replace the existing allergenic protein, but is used in conjunction with the existing allergenic protein, and is in a position to contribute to an increased selection of allergens to sensitize individual patients. Therefore, there is no need to consider the effect of the use of the novel allergenic protein of the present disclosure in comparison with those of the conventional allergenic proteins, and if the novel allergenic protein of the present disclosure has an allergenic activity that is clearly different from those of the conventional allergenic proteins, it can contribute to the diagnosis of an allergic disease such as CRD and to the treatment based on such diagnosis, which makes its industrial use valuable.

When the agent for preventing or treating an allergic disease caused by juniper pollen of the present disclosure is used as an agent for a desensitization treatment, it is preferable to provide the juniper pollen protein of the present disclosure as it is, or in a powder form by drying, or as a formulation by adding a commonly used adjuvant and various additives such as a stabilizer, an excipients, a dissolution aid, an emulsifier, a buffer, a soothing agent, a preservative, a coloring agent, etc., as needed, by a common method.

For example, the purified juniper pollen protein in a powder form can be dissolved in a physiological saline supplemented with phenol to be used as a stock solution of the antigen for the desensitization treatment.

The agent for preventing or treating an allergic disease caused by juniper pollen of the present disclosure can be administered topically or systemically by a common administration route, such as an oral or parenteral (transdermal, transmucosal, intradermal, subcutaneous, intramuscular, intraperitoneal, etc.) route. Examples of the dosage form applicable to these administration methods include a lozenge, a sublingual tablet, an injectable, ophthalmic drops, a nasal spray, a poultice, cream, and lotion.

A method for diagnosing an allergic disease caused by juniper pollen of the present disclosure can be carried out by performing topical or systemic administration by a common administration route, such as an oral or parenteral (transdermal, transmucosal, intradermal, subcutaneous, intramuscular, intraperitoneal, etc.) route. Examples of the dosage form applicable to these administration methods include a lozenge, a sublingual tablet, an injectable, ophthalmic drops, a nasal spray, a poultice, cream, and lotion.

The dosage and number of doses of the agent for preventing or treating an allergic disease caused by juniper pollen of the present disclosure depend on the route of administration, symptoms, etc. For example, they are appropriately selected to be in the range of about 0.1-1,000 μg per dose for an adult and administered from once to about several times a week.

Juniper pollen is also known as an allergen for pollinosis caused by mountain cedar pollen, which is a plant belonging to the Cupressaceae family that causes pollinosis in regions such as France, Italy, and Australia. The present disclosure is expected to be effective in the treatment of pollinosis caused by mountain cedar pollen as well as juniper pollen, Japanese cedar pollen, and Japanese cypress pollen.

It is known that the positivity rate of juniper antibody in Japanese patients with Japanese cedar pollinosis is high, and that the positivity rate of juniper antibody is especially high in patients with Japanese cypress pollinosis. The present disclosure can be used for the treatment of Japanese cedar pollinosis and Japanese cypress pollinosis in Japanese people and is expected to be effective.

### Diagnosis of Allergic Disease Caused by Juniper Pollen

The juniper pollen protein of the present disclosure can serve as an agent for diagnosing an allergic disease caused by juniper pollen and can be used to diagnose the allergic disease caused by juniper pollen.

Such an agent for diagnosing an allergic disease caused by juniper pollen can be used, for example, as a reagent for diagnosing skin reactions to the allergic disease caused by juniper pollen, i.e., a reagent for an intradermal test, a prick test, etc. In addition, a reagent can be prepared for a specific IgE antibody assay for diagnosis using a patient's serum or plasma. (Practical Guideline for the Management of Allergic Rhinitis in Japan <PG-MARJ>, 2013 Edition, Life Science Co., Ltd.)

When used as a reagent for diagnosing skin reactions, the juniper pollen protein of the present disclosure obtained by the method described above is, for example, dried to a powder form, and dissolved and diluted upon use in a physiological saline solution containing phenol or glycerin. When used as a reagent for a specific IgE antibody assay, IgE antibodies in a patient sample are allowed to bind to the juniper pollen protein of the present disclosure in an aqueous phase or on a solid phase, and detection is performed by, but not limited to, a method based on the principle of fluoro-enzyme immunoassay, chemiluminescent enzyme immunoassay, enzyme immunoassay, etc.

Thus, the present disclosure provides a method for detecting a skin reaction, the method comprising reacting the juniper pollen protein with a sample. The present disclosure also provides a kit for detecting a skin reaction, the kit comprising the juniper pollen protein. Herein, the term “reacting” includes both contacting the juniper pollen protein of the present disclosure with a patient's sample and binding IgE antibody in the sample to the juniper pollen protein of the present disclosure.

When the juniper pollen protein of the present disclosure is used as a kit, the protein can be combined with other solvent and solute to make a composition. For example, it can be combined with distilled water, a pH buffering reagent, a surfactant, and the like. The juniper pollen protein can also be labeled with an enzyme or biotin for use. As the labeling enzyme, HRP (horseradish peroxidase), alkaline phosphatase, malate dehydrogenase, α-glucosidase, β-galactosidase, gold colloid, or the like can be used.

When the present disclosure is used for a kit, the above-mentioned solvent, solute, enzyme-labeled reagent, substrate solution, reaction stopping solution, washing solution, and instructions for use, etc. can be included in addition to the protein of the present disclosure.

### Antibody Against Juniper Pollen Protein

Antibodies against the juniper pollen protein of the present disclosure are antibodies that can bind specifically to the juniper pollen protein of the present disclosure. The above antibodies refer to immunoglobulins (IgA, IgD, IgE, IgG, IgM, and antigen-binding fragments thereof (Fab fragment, F (ab′) 2 fragment, Fc fragment, or scFv, etc.)), and examples thereof include, but are not limited to, polyclonal antibodies, monoclonal antibodies, single-chain antibodies, anti-idiotypic antibodies, and humanized antibodies.

The above antibodies can be produced using various known methods, and the methods of their production are not particularly limited.

For example, as a method of obtaining a monoclonal antibody of the present disclosure, first, a non-human mammal is immunized with the juniper pollen protein or a partial peptide thereof, and then antibody-producing cells (e.g., B cells) are harvested from the immunized animal. The antibody-producing cells are fused with myeloma cells to generate a hybridoma (fusion cell line). An antibody produced from the hybridoma is then harvested to obtain the monoclonal antibody of interest.

The amino acid sequence based on which the antigen peptide is synthesized is a sub-sequence of the full-length amino acid sequence of the juniper pollen protein, and an amino acid sequence of any continuous length of about 10-50 amino acids is selected therefrom. The partial peptide can be synthesized by a method known to those skilled in the art, such as the Fmoc or tBoc method.

Examples of the type of the non-human mammal to be immunized include, but are not particularly limited to, mouse, rat, guinea pig, rabbit, dog, and goat, with mouse being preferred. The total dose of antigen is 10-150 μg per animal. When immunizing with an antigen, it is common to mix the antigen solution with an adjuvant. Examples of the type of the adjuvant include Freund's complete adjuvant (FCA), Freund's incomplete adjuvant (FIA), and aluminum hydroxide adjuvant. Immunization is performed mainly by intravenous, subcutaneous, intraperitoneal, intramuscular, or subcutaneous footpad injection. Moreover, the interval between immunizations is not particularly limited, and immunizations are performed 1-10 times at intervals of a few days to several weeks, preferably 2-3 weeks.

The antibody-producing cells are prepared from spleen cells, etc. or a regional lymph node, etc. of the immunized non-human mammal.

Cell fusion is a process of fusing the above antibody-producing cells with myeloma cells to produce cells (hybridoma) that proliferate semi-permanently while producing antibodies. A person skilled in the art can fuse antibody-producing cells with myeloma cells using a known cell fusion method.

Next, a hybridoma that produces the antibody of interest is selected from the cells after the cell fusion treatment. 10-14 days after the cell fusion, the selected cells form colonies in a HAT medium. The culture supernatant in each well of the colony-positive culture plate is collected to check the antibody titer against the juniper pollen protein by ELISA or the like.

Cells in the wells that are finally selected are cloned to obtain single cells. For cloning, for example, the cell suspension is diluted appropriately in a RPMI 1640 medium containing 10-20% FCS and the cells are seeded one cell per well in a 96-well culture plate. After cell seeding, the culture supernatant is collected from the colony-positive wells. The cells in the selected wells are further increased to some extent to establish hybridoma lines. Cloning may be done several times if necessary.

A monoclonal antibody specific to the juniper pollen protein is purified and harvested from the established hybridoma line. Specifically, there are a method of preparing an antibody from a culture supernatant cultured in a medium having a low serum concentration, a method of preparing an antibody from a culture supernatant cultured in a commercially available serum-free medium, a method of injecting the hybridoma into an abdominal cavity of an animal, collecting the ascites fluid, and preparing an antibody from the ascites fluid, and other methods.

The epitope (antigenic determinant) of the antibody against the juniper pollen protein of the present disclosure is not limited as long as it is at least a part of the antigen, i.e., juniper pollen protein.

The antibody against the juniper pollen protein is also not limited to the anti-juniper pollen protein monoclonal antibody itself produced by the above hybridoma, but all antibodies that bind to an epitope recognized by the monoclonal antibody produced by the hybridoma are encompassed by the antibody of the present disclosure. The term “epitope” here refers to an epitope recognized by the monoclonal antibody produced by the above hybridoma.

The monoclonal antibody of the present disclosure can also be a gene recombinant antibody or an antigen-binding fragment that is prepared and expressed by a recombination process. For example, the antibody of the present disclosure may be a chimeric antibody, a humanized antibody, or a fully human antibody. A recombinant antibody of the present disclosure can be produced by recombinant expression of the heavy and light chains.

For expression of the gene recombinant antibody, for example, a recombinant expression vector having nucleic acids encoding the heavy and light chains of the antibody is introduced into a host cell, and the host cell into which said vector has been introduced is cultured. Then, the antibody of interest is recovered from the culture of the host cell. To obtain the genes for the heavy and light chains of the antibody, incorporate these nucleic acids into an expression vector, and introduce the expression vector into a host cell, a recombination method standard in the art (Sambrook et al., Molecular Cloning: A Laboratory Manual, Fourth Edition, Cold Spring Harbor Laboratory Press, 2012) can be employed.

Moreover, in a method for obtaining a polyclonal antibody of the present disclosure, a non-human mammal is immunized with a juniper pollen protein or a partial peptide thereof, and blood is collected after 3-4 immunizations to measure the antibody titer by ELISA or the like. After confirming that the antibody titer has increased sufficiently, whole blood is collected to separate and purify the antibody by a conventional method or the like.

The above antibody can be used, for example, to identify an organism or a tissue or cell thereof that expresses the juniper pollen protein of the present disclosure. For example, the antibody can be used to measure the presence or absence of the juniper pollen protein in the air, indoor space or human mucosa. The measurement can be performed by a known immunological method, for example, by ELISA.

Since the juniper pollen protein is an allergen for Japanese cypress pollinosis, a juniper pollen protein can be detected by reacting the antibody of the present disclosure with a sample and measuring the juniper pollen protein in the sample.

Accordingly, the present disclosure provides a method for detecting a juniper pollen protein, the method comprising detecting a juniper pollen protein by reacting the antibody of the present disclosure or a fragment thereof with the juniper pollen protein.

In the present disclosure, an antibody against the juniper pollen protein or a fragment thereof can be used as a reagent or kit for detecting the juniper pollen protein. The kit of the present disclosure is applicable in the same way as when the juniper pollen protein is used as a kit.

Hereinafter, the present disclosure will be described more specifically by means of examples. The present disclosure, however, should not be limited to these examples.

### EXAMPLES

### Example 1

### Purification of Juniper Protein

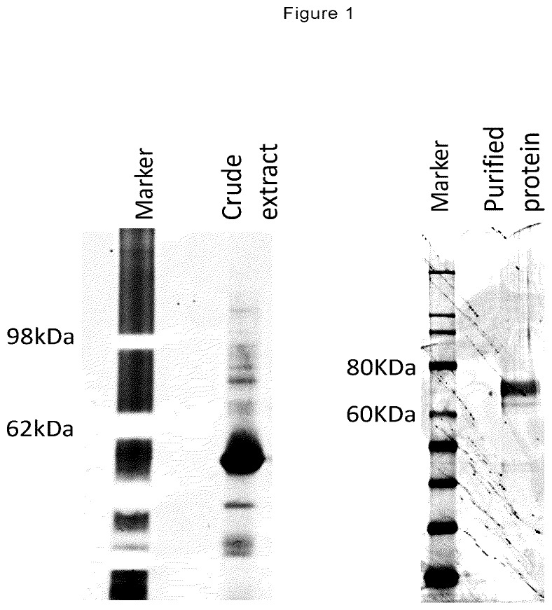

To 5 g of juniper pollen (Juniperus ashei), 200 mL of extraction buffer (25 mM Tris buffer, pH 8.0) was added. The resultant was disrupted with an ultrasonic disruptor (UD-201, TOMY) and centrifuged (10,000×g, 10 minutes) to collect the supernatant. The supernatant was added to Vivapure Q Maxi H (Sartorius) equilibrated with 25 mM Tris buffer (pH 8.0) and the non-adsorbed fraction was collected. This was added to Vivapure S Maxi H (Sartorius) equilibrated with 20 mM acetate buffer (pH 5.2) for adsorption, washed with 20 mM acetate buffer containing 0.35 M NaCl (pH 5.2), and then eluted with 20 mM acetate buffer containing 0.5 M NaCl (pH 5.2). The result of SDS-PAGE of the purified juniper protein is shown in FIG. 1.

### Example 2

### Evaluation of Allergenic Activity of Purified Juniper Protein

### (i) Lymphocyte Proliferative Response to Purified Juniper Protein

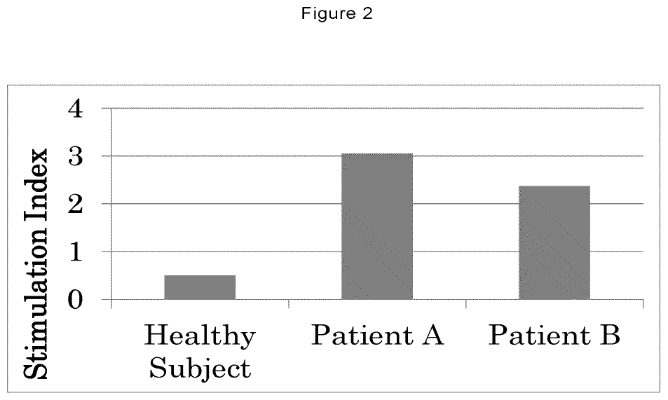

Peripheral blood mononuclear cells were isolated from 30 mL of peripheral blood obtained from two subjects with tree allergy symptoms in the spring season and one healthy subject, and suspended in a RPMI-1640 medium containing 10% inactivated autologous plasma to 1×10⁶ cells/mL. 180 μL of the prepared cell suspension and 20 μL of purified juniper protein solution (concentration 100 μg/ml) were seeded into a 96-well plate (2×10⁵ cells/well). After 3 days of cultivation under the conditions of 37° C. and 5% CO₂, 3H-thymidine was added, and cell proliferation was assessed by measuring the amount of uptake as radioactivity up to 16 hours later. Stimulation index was calculated by dividing the amount of 3H-thymidine uptake when the purified juniper protein was added by the amount of uptake when it was not added, and the results are shown in FIG. 2.

In general, a stimulation index of 1.8 or higher is considered the positive criterion in a lymphocyte stimulation test for clinical examination (Uchida, Shigeyuki et al., Kanzo, vol. 30, no. 4, 439-443, 1989). Based on this criterion, two out of two subjects with allergy symptoms (100%) tested positive. On the other hand, the healthy subject was tested negative. These results indicated that sensitization to the protein of the present disclosure at the T-cell level was observed at a high frequency in individuals with tree allergy symptoms in the spring season.

### (ii) Activation of Basophils by Purified Juniper Protein

CD203c expression is known to be enhanced in basophils stimulated and activated by an allergen (De Weck, A L. et al., Int. Arch. Allergy Immunol., 146:177-189, 2008). Allergenicity kit based on this principle (Beckman Coulter) was used to analyze basophil activation in the blood of a subject with tree allergy symptoms in the spring season or a healthy subject.

Specifically, 20 μL of the purified juniper protein solution of the present disclosure was added to 100 μL of whole blood collected with heparin to a final concentration of 100 ng/mL, while 20 μL of PBS (−) was added to a negative control sample. Then, 20 μL of antibody cocktail containing CD3-PC7, CRTH2-FITC, and CD203c-PE was added to all samples and incubated at 37° C. for 15 minutes. 100 μL of reaction stopping solution and 2 mL of lysing fixative solution were added and allowed to react for 10 minutes at room temperature. After centrifugation (200×g, 5 minutes), the supernatant was removed and 3 mL of phosphate buffer solution (PBS) was added and centrifuged again.

After removal of the supernatant, cells were suspended in 0.1% formaldehyde-supplemented PBS and measured by flow cytometer (FACSVerse, BD Biosciences). Based on the data obtained, the basophils in the blood cells were sorted using CD3-PC7 negativity and CRTH2-FITC positivity as indicators, and the intensity of CD203c expression was analyzed. The data shows the proportion (%) of CD203c-positive basophils in the samples stimulated with the protein solution of the present disclosure minus the proportion (%) of CD203c-positive basophils in each negative control sample, and values greater than or equal to 1.5, i.e., twice the maximum value of 0.75% in healthy subjects, were considered positive.

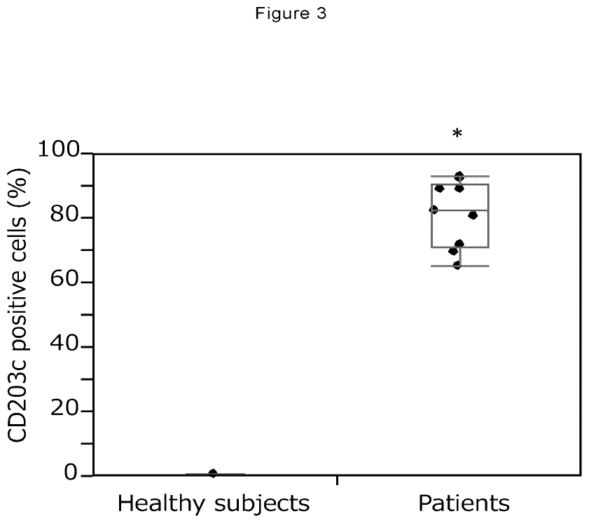

As a result, as shown FIG. 3, when the purified juniper protein was added, CD203c expression was not enhanced in two out of two healthy subjects, whereas CD203c expression was enhanced in nine out of nine subjects (100%) who exhibited tree allergy symptoms in the spring season, and the difference was significant (p<0.05, Welch's t-test). Thus, subjects with tree allergy symptoms in the spring season have IgE that binds to the purified juniper protein of the present disclosure at a high frequency, further indicating that the protein of the present disclosure has an allergenic activity.

### Example 3

### Determination of Partial Internal Amino Acid Sequences of Purified Juniper Protein

To reveal partial amino acid sequences in the protein of the present disclosure, a peptide mixture obtained by trypsinolysis of the purified juniper protein was analyzed. After SDS-PAGE of the purified juniper protein, a protein band to be analyzed was cut out from the SDS gel. From the cut gel slice, a peptide mixture was obtained by trypsin hydrolysis. The peptide mixture was analyzed by LC-MS/MS, thereby obtaining peptides represented by LPLLAR [partial amino acid sequence 1 (SEQ ID NO:3)], WIVDETTGLR [partial amino acid sequence 2 (SEQ ID NO:4)], ATVGETFAR [partial amino acid sequence 3 (SEQ ID NO:5)], YFNPNTWVK [partial amino acid sequence 4 (SEQ ID NO: 6)], and IRNPDFIAR [partial amino acid sequence 5 (SEQ ID NO:7)].

### Example 4

### Determination of Nucleotide Sequence of Purified Juniper Protein

### <Extraction of Juniper Total RNA>

To 2 g of frozen juniper pollen, 25 mL of Plant RNA Isolation Reagent (Invitrogen) was added, mixed, and allowed to stand at room temperature for 5 minutes. After centrifugation (2,600×g, 5 minutes) at 4° C., the upper layer was filtered through a mesh with a mesh size of 100 μm by decanting. Then, to the collected filtrate, ⅕ volume of 5M NaCl and ⅗ volume of chloroform were added. After centrifugation (2,600×g, 30 minutes) at 4° C., 20 mL of the upper layer was collected, and 9/10 volume of isopropyl alcohol was added and stirred. The resultant was allowed to stand at room temperature for 10 minutes and then RNA was precipitated by centrifugation (2,600×g, 30 minutes) at 4° C. After removing the supernatant, 10 mL of 75% ethanol was added to the RNA pellet for washing. After further centrifugation at 4° C. (2,600×g, 5 minutes), the supernatant was removed and the total RNA was dried at room temperature and dissolved in 200 μL of RNase-free water.

RNA was purified from the total RNA solution using RNeasy Mini kit (Qiagen) by the following steps. After adding 200 μL of 70% aqueous ethanol solution to the total RNA solution, the mixture was added to RNeasy Mini column and centrifuged (10,000 rpm, 15 seconds). Furthermore, the column was washed with 350 μL of buffer RW1 and treated with DNase using RNase-Free DNase set (Qiagen). After washing the column with 350 μL of buffer RW1, the column was washed twice with 500 μL of buffer RPE. After washing, 30 μL of RNase-free H₂O was added and the resulting solution was collected as cleaned-up total RNA.

<Preparation of Juniper cDNA>

From the resulting cleaned-up total RNA, cDNA was synthesized using Superscript IV First-Strand Synthesis System for RT-PCR (Invitrogen). 2 μg of the total RNA was dissolved in 11 μL of purified water, to which 1 μL of 50 μM oligo (dT) 20 and 1 μL of 10 mM dNTPs were added and incubated at 65° C. for 5 minutes. The sample was cooled on ice, to which 4 μL of 5×SSIV buffer, 1 μL of 100 mM DTT, 1 μL of ribonuclease inhibitor, and 1 μL of SuperScript IV reverse transcriptase were added and mixed. After incubation at 50° C. for 10 minutes, the reaction was stopped by a treatment at 80° C. for 10 minutes. Subsequently, 1 μL of RNase H was added to the sample and incubated at 37° C. for 20 minutes to obtain juniper cDNA.

### <Cloning>

Using juniper cDNA as a template, Primer 1 (ATGACGATGGCGGCGCTA: SEQ ID NO:8) as a sense primer, and Primer 2 (TCAAAGTTGATGCAACAATTGTTTGTTG: SEQ ID NO:9) as an antisense primer, PCR was performed with KOD Fx Neo (TOYOBO). Composition of PCR reaction solution: 10 μL of 5× PrimeSTAR GXL Buffer, 4 μL of dNTP mixture (2.5 mM each), 0.5 μL of cDNA, 1.5 μL each of 10 mM primer, 1 μl of PrimeSTAR GXL DNA polymerase, and up to 50 μl of sterile purified water.

Cycle conditions: 98° C. for 10 seconds, 68° C. for 2 minutes (35 cycles)

The PCR product was purified with Wizard SV Gel and PCR Clean-Up System (Promega). The purified PCR product was incorporated into pCR 2.1-TOPO TA vector using TOPO TA Cloning Kit for Sequencing (Thermo Fischer).

The nucleotide sequence was analyzed from the obtained vector to obtain sequence information.

For further analysis of the 3′-sequence, juniper cDNA with an anchor sequence added to the 3′ end was generated using 5′/3′ RACE kit, 2nd generation (Roche). Nested PCR was performed with KOD Fx neo using two-step primers, Primer 3 (AGTGGATGTGTTTAGATGGG: SEQ ID NO:10) and Primer 4 (AGCAGTGGTTTAAAGTCATAA: SEQ ID NO:11).

The PCR product was purified with Wizard SV Gel and PCR Clean-Up System (Promega). The purified PCR product was incorporated into pCR 2.1-TOPO TA vector using TOPO TA Cloning Kit for Sequencing (Thermo Fischer). The nucleotide sequence of the resulting vector was analyzed to obtain the 3′-terminal sequence.

The cDNA encoding the juniper protein of the present disclosure was encoded by the polynucleotide represented by SEQ ID NO:1. The amino acid sequence (SEQ ID NO: 2) encoded by SEQ ID NO:1 contained partial amino acid sequences 1-5, indicating that the protein of the present disclosure was a protein encoded by the polynucleotide represented by SEQ ID NO:1.

### Sequence Listing Free Text

SEQ ID NOS: 1-7: Synthetic peptides
SEQ ID NOS: 8-11: Synthetic DNAs

1. A juniper pollen protein selected from (a)-(c) below:
(a) a protein comprising the amino acid sequence represented by SEQ ID NO:2;
(b) a protein comprising the amino acid sequence represented by SEQ ID NO:2 with one or several amino acids substituted, deleted or added, and having a juniper pollen allergenic activity; and
(c) a protein comprising an amino acid sequence having 90% or more identity to the amino acid sequence represented by SEQ ID NO:2, and having a juniper pollen allergenic activity.

2. A polynucleotide encoding a juniper pollen protein selected from (a)-(c) below:
(a) a protein comprising the amino acid sequence represented by SEQ ID NO:2;
(b) a protein comprising the amino acid sequence represented by SEQ ID NO:2 with one or several amino acids substituted, deleted or added, and having a juniper pollen allergenic activity; and
(c) a protein comprising an amino acid sequence having 90% or more identity to the amino acid sequence represented by SEQ ID NO:2, and having a juniper pollen allergenic activity.

3. A polynucleotide selected from (d)-(f) below:
(d) a polynucleotide comprising the nucleotide sequence represented by SEQ ID NO: 1;
(e) a polynucleotide that hybridizes with a polynucleotide comprising a nucleotide sequence complementary to the nucleotide sequence represented by SEQ ID NO: 1 under stringent conditions and that encodes a protein having a juniper pollen allergenic activity; and
(f) a polynucleotide that comprises a nucleotide sequence having 90% or more identity to the nucleotide sequence represented by SEQ ID NO: 1 and that encodes a protein having a juniper pollen allergenic activity.

4. A recombinant vector, comprising:
the polynucleotide of claim 3.

5. A transformant, comprising:
the recombinant vector of claim 4.

6. A method for producing a juniper pollen protein, the method comprising:
culturing the transformant of claim 5; and
collecting the juniper pollen protein from the resulting culture.

7-9. (canceled)

10. A kit for diagnosing an allergic disease caused by juniper pollen, the kit comprising:
the juniper pollen protein of claim 1 as an active ingredient.

11. A method for detecting an allergic disease caused by juniper pollen, the method comprising:
reacting the juniper pollen protein of claim 1 with a sample.

12. (canceled)

13. An antibody against the juniper pollen protein according to claim 1.

14. The antibody against the juniper pollen protein according to claim 13, wherein the antibody is a monoclonal antibody.

15-18. (canceled)

19. A method for preventing or treating an allergic disease caused by juniper pollen, the method comprising:
administering the juniper pollen protein of claim 1 to a patient.

20. A method for diagnosing an allergic disease caused by juniper pollen, the method comprising:
administering the juniper pollen protein of claim 1 to a patient.


SEQUENCE LISTING (11 sequences)

SEQ ID NO: 1 - DNA, 1710 bp, Juniperus ashei
  source 1..1710: mol_type = other DNA
  CDS 1..1710
atgacgatggcggcgctacgattgctaacaacgttgctattgctgctactggcagcactggcacattcgttgccgctactggcacgtggcaggtggatcgtggacgagacgacggggctcagagtaaagctagcatgcgtaaactgggtgggccacctggagcccggccttcccgaaggcctcaaccggttgccggtcaccaccgtagctcagaccattagctctctgggcttcaattgcgtgcgcctcacctactccatccacatgctcaccagggacacctacacgaaggcgacggttggggagacttttgcccggctcaatctcaccgaggcggcctcaggaatcgagcacaacaacccagggttgctcggcttgagccacgtagccgcatatcatcgcgtcgtggcggcgctatcggaggcgggtgtcatggtcatcttggacaaccatgtgagcaagcccaagtggtgttgcgctgtcgatgacggcaacgcgttcttcggcgaccgttacttcaaccccaacacgtgggtcaaagggctcggtctcatggccacatacttcaacaacacgcccaacatcatcgccatgtcgcttcgcaacgagctccgtggcaaccaatcgaccccgatatcatggtccaagcacatgcaatggggtgccgcaaccgtccacaaggccaacccgaaagtcctcgtggtcctctcagggctgcactacgacaccgacctcagcttcttaccggtcctaccggtcaccttacctttcaaggacaaaattgtctacgaagggcattggtactcctttggcatcccgtggcacgctggcctccctaaccacgtctgtacaaacgagacagcacgatttaagaggaatatcgggtttgttacattgtcagctaatggtactgcggcacctctttttataagcgagtttgggatcgaccagcgatacgtgaatgacaatgacaacagatatttgaactgtatattggcttatctggccgaggaggacttggactgggcgctgtggacaatgggagggagctattattaccggtcagaccagacgcccgttaaagacttcgaggaaacatatgggtttttcaatcacgactggtctcgcatcagaaatcctgactttattgctcggctcaaggaaatacaacagcccattcaagacccatacttatctccagggccatattatcagataatataccacccagcatcaggtctatgtatagaatccagcataggaaacacaattcatctgggatcatgccaaagtgtgaaaagcagatggaactatgatgccactgtggaaggccccattgggctgatgggaacttcatcctgcattaccactcaaggagatgggctgcctgcaattataactgagaattgctcagcacccaacaacacaatgtggagcacagtttccactgcacagctgcagctggccaccagagtttctgatggagagggaaaagagaagtggatgtgtttagatgggagtaaaagtcctctcattttaacaacagaatgcatttgcatcactgactctcactgctacccaaaactcaatccagaaaagcagtggtttaaaagtcatcgctaccaacaaacaattgttgcatcaactttgattcttgtattggttttttttcccaattttgtgatgtatttcattcctctccaattgtaa

SEQ ID NO: 2 - protein, 569 aa, Juniperus ashei
MTMAALRLLTTLLLLLLAALAHSLPLLARGRWIVDETTGLRVKLACVNWVGHLEPGLPEGLNRLPVTTVAQTISSLGFNCVRLTYSIHMLTRDTYTKATVGETFARLNLTEAASGIEHNNPGLLGLSHVAAYHRVVAALSEAGVMVILDNHVSKPKWCCAVDDGNAFFGDRYFNPNTWVKGLGLMATYFNNTPNIIAMSLRNELRGNQSTPISWSKHMQWGAATVHKANPKVLVVLSGLHYDTDLSFLPVLPVTLPFKDKIVYEGHWYSFGIPWHAGLPNHVCTNETARFKRNIGFVTLSANGTAAPLFISEFGIDQRYVNDNDNRYLNCILAYLAEEDLDWALWTMGGSYYYRSDQTPVKDFEETYGFFNHDWSRIRNPDFIARLKEIQQPIQDPYLSPGPYYQIIYHPASGLCIESSIGNTIHLGSCQSVKSRWNYDATVEGPIGLMGTSSCITTQGDGLPAIITENCSAPNNTMWSTVSTAQLQLATRVSDGEGKEKWMCLDGSKSPLILTTECICITDSHCYPKLNPEKQWFKSHRYQQTIVASTLILVLVFFPNFVMYFIPLQL

SEQ ID NO: 3 - protein, 6 aa, synthetic construct
  REGION 1..6: note = Synthetic peptide
LPLLAR

SEQ ID NO: 4 - protein, 10 aa, synthetic construct
  REGION 1..10: note = Synthetic peptide
WIVDETTGLR

SEQ ID NO: 5 - protein, 9 aa, synthetic construct
  REGION 1..9: note = Synthetic peptide
ATVGETFAR

SEQ ID NO: 6 - protein, 9 aa, synthetic construct
  REGION 1..9: note = Synthetic peptide
YFNPNTWVK

SEQ ID NO: 7 - protein, 9 aa, synthetic construct
  REGION 1..9: note = Synthetic peptide
IRNPDFIAR

SEQ ID NO: 8 - DNA, 18 bp, synthetic construct
  misc_feature 1..18: note = Synthetic DNA
  source 1..18: mol_type = other DNA
atgacgatggcggcgcta

SEQ ID NO: 9 - DNA, 28 bp, synthetic construct
  misc_feature 1..28: note = Synthetic DNA
  source 1..28: mol_type = other DNA
tcaaagttgatgcaacaattgtttgttg

SEQ ID NO: 10 - DNA, 20 bp, synthetic construct
  misc_feature 1..20: note = Synthetic DNA
  source 1..20: mol_type = other DNA
agtggatgtgtttagatggg

SEQ ID NO: 11 - DNA, 21 bp, synthetic construct
  misc_feature 1..21: note = Synthetic DNA
  source 1..21: mol_type = other DNA
agcagtggtttaaagtcataa